(19) United States
(12) Patent Application Publication
(10) Pub. No.: US 2016/0086330 A1
(43) Pub. Date: Mar. 24, 2016

(54) METHOD AND APPARATUS FOR DETERMINING A POSITION OF AN OBJECT FROM MRI IMAGES

(71) Applicant: SIEMENS AKTIENGESELLSCHAFT, MUNICH (DE)

(72) Inventors: DAVID GRODZKI, ERLANGEN (DE); ANNEMARIE HAUSOTTE, ERLANGEN (DE); BJOERN HEISMANN, ERLANGEN (DE); ARNE HENGERER, MOEHRENDORF (DE); MARK ALEKSI KELLER-REICHENBECHER, SANDHAUSEN (DE); SEBASTIAN SCHMIDT, WEISENDORF (DE)

(73) Assignee: SIEMENS AKTIENGESELLSCHAFT, MUNICH (DE)

(21) Appl. No.: 14/858,479

(22) Filed: Sep. 18, 2015

(30) Foreign Application Priority Data
Sep. 19, 2014 (DE) 102014218924.1

(51) Int. Cl. G06T 7/00; A61B 5/11; A61B 5/113; A61B 5/00; G06T 19/20; G06T 7/20

(52) U.S. Cl. CPC G06T 7/0012; G06T 19/20; G06T 7/0044; G06T 7/204; A61B 5/113; A61B 5/4839; A61B 5/1128; A61B 5/1102; G06T 2200/08; G06T 2207/10021; G06T 2207/10028; G06T 2207/30096; A61B 2019/5236

(57) ABSTRACT

Periodic movement of an object, for example a tumor, is determined on the basis of 4D MRI images. A radiation source can be controlled (guided) as a function of the periodic movement of the object, thus enabling the compilation of a more efficient treatment plan with respect to time and radiation.

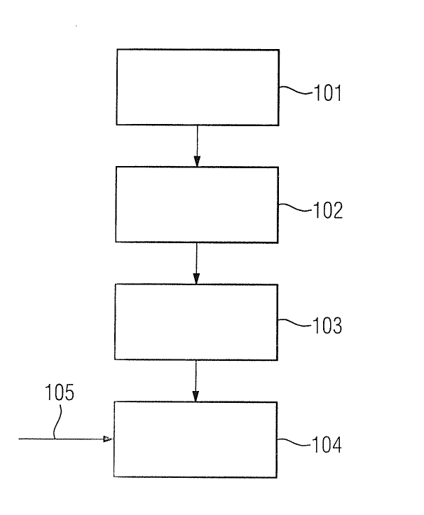

### BACKGROUND OF THE INVENTION

1. Field of the Invention

The invention concerns a method for determining a position of an object from MRI images, and an associated device and system and a corresponding computer-readable storage medium.

2. Description of the Prior Art

Magnetic resonance imaging (MRI, also known as MR) is an imaging modality specifically used in medical diagnostics for the representation of the structure and functions of tissues and organs in the body. It is based on the principles of nuclear magnetic resonance and is therefore also referred to as nuclear magnetic resonance tomography. General details can be found, for example, at de.wikipedia.org/wiki/Magnetresonanztomographie.

Computed tomography (abbreviated CT) is an imaging modality used in radiology. Details can be found, for example, at de.wikipedia.org/wiki/Computertomographie.

Radiotherapy is an attempt to destroy malignant tissue selectively with ionizing radiation. Such malignant tissue is, for example, irradiated directly from outside the body with X-rays or (heavy) ions. Alternatively, it is possible to implant radioactive radiation sources, known as seeds.

Radiotherapy is planned before the start of treatment so that the diseased tissue is irradiated as effectively as possible and, if possible, healthy tissue is not irradiated at all. Planning of this kind is performed, for example, with three-dimensional imaging using MRI or CT. The three-dimensional images are first used to select volumes to be irradiated, then a dose is established for each volume and this information is used to calculate a radiotherapy plan for controlling a linear accelerator.

For respiratory-system tumors (for example in the lungs or liver), the movement of the tumor volume presents an additional problem: depending upon the course of the respiration, the position of the volume to be irradiated rises or drops by a several millimeters or centimeters.

Known solutions have the disadvantage that non-diseased tissue is also exposed to radiation or that the treatment takes a long time overall.

### SUMMARY OF THE INVENTION

An object of the invention is to avoid the aforementioned disadvantages and in particular to provide an efficient possibility for the determination of the position of a target object, for example malignant tissue.

This object is achieved by a method in accordance with the invention for determining a position of an object, wherein multiple temporally successive MRI images are compiled, and a periodic movement of the object is determined in a processor on the basis of the compiled multiple MRI images.

An MRI image is an image compiled by operation of a magnetic resonance imaging as an imaging apparatus according to an imaging protocol. The periodic movement of the object can be a movement of the object that is cyclical, repetitive or recurrent in some other way. The determination of the movement of the object can be a determination of a change to the size, location or alignment or position of the object.

Here, it is of advantage that magnetic resonance imaging is used for the compilation of, in particular, 4D images and the (periodic) movement of the object can be used to achieve efficient setting or guidance of a radiation source, for example onto the object or a part of the object. This enables, for example, time-efficient irradiation of the object or ensures that substantially only the object and no other tissue is irradiated.

In an embodiment, the periodic movement is determined on the basis of respiration.

For example, the period of the movement can be based on a respiratory period (respiratory rate).

In another embodiment, the periodic movement is determined on the basis of a heartbeat.

In an embodiment, the periodic movement is determined on the basis of a pattern recognition performed on the basis of the MRI images.

For example, at least one state of the periodic movement (for example maximum inspiration or expiration) can be established with reference to the pattern recognition by identifying, for example, when a lung volume is maximum or minimum. In particular, the MRI images used for the pattern recognition can be images (two-dimensional or three-dimensional) with high contrast or clearly identifiable patterns.

In another embodiment, the object is a tumor.

In a further embodiment, the object is a spherical or ellipsoidal region, or includes such a region, or lies within such a region.

In another embodiment, a radiation source is controlled as a function of the periodic movement of the object.

The radiation source can be a linear accelerator that emits X-rays or (heavy) ion rays. The radiation source is advantageously guided according to the movement of the object. In this case, both the actual radiation source or the beam emitted by the radiation source is controlled or guided (for example deflected).

In another embodiment, temporally successive MRI images are used to compile three-dimensional images of the object and multiple three-dimensional images respectively obtained over time are combined to form a four-dimensional image of the object.

In another embodiment, the periodic movement of the object is determined taking into account at least one of the following changes:

a change of the size of the object,

a change of the location and alignment of the object,

a change of the position of the object.

Hence, it is possible, for example, to take account of any combination of a change to the size, location or alignment or position of an object during the course of the movement of the object. For example, during a period of respiration, the object can be intermittently compressed or contorted. The position of the object can also be displaced by the actual respiration.

The explanations relating to the method are also applicable to the other claim categories as appropriate.

The aforementioned object is also achieved by a device for determining a position of an object having a processor configured to compile multiple temporally successive MRI images and to determine a periodic movement of the object from the compiled multiple MRI images.

The processor can be at least partially hard-wired or a logic circuit configuration configured, for example, such that the method described herein can be performed. The processor can be or include any type of processor or calculator or computer with the appropriate necessary peripherals (memory, input/output interfaces, input-output devices, etc.).

The above explanations relating to the method apply correspondingly to the device. The device can be embodied as one component or be divided into multiple components.

The aforementioned object is also achieved a system having at least one of the devices described herein.

In an embodiment, the processor is configured such that a radiation source can be controlled as a function of the periodic movement of the object.

The invention also encompasses a system having a further device with a further processor, which is configured such that a radiation source can be controlled in dependence on a periodic movement of an object, wherein the periodic movement can be provided by the aforementioned device.

The invention also encompasses a computer-readable data storage medium, encoded with computer-executable instructions (for example in the form of a program code) that cause the method described above to be implemented when the instructions are executed by a computer or processor in which the storage medium is loaded.

### BRIEF DESCRIPTION OF THE DRAWING

The single FIGURE is a flowchart of basic steps of the method for planning irradiation and carrying out irradiation in accordance with the invention.

### DESCRIPTION OF THE PREFERRED EMBODIMENTS

In the embodiment used to explain the invention, it is proposed to plan radiotherapy for a tumor, for example a lung tumor, on the basis of a four-dimensional (4D) imaging method. The imaging method used for the 4D imaging method is, for example, magnetic resonance imaging (MRI).

For example, a rapid MRI sequence is used to compile a sequence of 2D images and this sequence is assembled into a 3D image. A temporal sequence of 3D images produces the 4D image. Hence, it is possible to record a 3D volume with high time resolution, for example a few seconds per image, over a period of from, for example, one minute to several minutes.

The rapid MRI sequence can be generated, for example, using a 4D cine method (see A. C. Larson et al.: Self-Gated Cardiac Cine MRI; Magn Res Med. January 2004 ; 51(1): 93-102), an URGE method (see O. Heid et al.: Ultra-Rapid Gradient Echo Imaging; MRM 33:143-149 (1995)) or a so-called “compressed sensing k-t” method (see J. Tsao et. al.: k-t BLAST and k-t SENSE: Dynamic MRI With High Frame Rate Exploiting Spatiotemporal Correlations, Magnetic Resonance in Medicine 50:1031-1042 (2003)).

Correspondingly, a 4D data record is generated over time (as a fourth dimension) that may be used to determine an irradiation volume. The irradiation volume is a volume, for example an approximately spherical or ellipsoidal volume, which, for example, at least partially defines an area to be irradiated. Preferably, the tumor is enclosed by this area or the area lies (at least partially) within the tumor.

For example, a temporal maximum intensity projection (t-MIP) can be compiled specifying an irradiation volume of the tumor.

Optionally, it is possible to determine a movement taking into account, for example, a periodic movement (with a respiratory rate) of at least one organ, for example the lungs, caused by the respiration of the patient. For example, the temporally periodic course of the deflection of the organ can be determined, i.e. modeled on the basis of the patient's respiration and, on the basis of a model of this kind, a future deflection of the organ can be predicted with a high degree of precision. This enables targeted irradiation at a place at which the organ (or the tumor) will be located during the course of the periodic deflection caused by the respiration.

For example, the tumor can be assumed to be a round object in the data record of the 4D data. The periodic movement of the respiration causes this round object to be moved from its resting position. This movement can be modeled as described above. A fit algorithm can be used to determine the size of the object, its central position and its deflection. The irradiation volume and/or the position of the irradiation can be planned on the basis of information of this kind.

Optionally, 4D images (time-resolved images of the volume) can be assigned to a respiratory state with reference to a pattern recognition. For example, the size (change) to the lungs over time can indicate whether the patient is currently breathing in or out or when the respective breathing in or out has finished. Recognized and evaluated patterns of this kind can be assigned to a respiratory state and used for improved (i.e. more precise) irradiation. In particular, it is possible to use pattern recognition of this kind to improve modeling of the movement of the organ or tumor.

For example it is possible for the respiratory state to be determined at a transition with high image contrast, such as a transition from tissue to air at the pulmonary borders, by means of the pattern recognition.

Hence as a result, it is possible to determine an irradiation plan as a function of the respiration, i.e. the position of the tumor during the course of the respiration (breathing position). This information can be used to control an irradiation unit (for example a linear accelerator). For example, the linear accelerator receives the information for the determination of the breathing position from another device, for example a breathing belt or an optical system. This enables the breathing position determined, for example from the breathing belt, to be used, on the basis of the modeling of the tumor and the respiration, to guide the irradiation during the course of the periodically repeating respiration according to the movement of the tumor during the course of the respiration.

For example, the breathing belt is used to determine the respiratory rate of the patient; the periodic movement of the tumor over time can be determined on the basis of the respiratory rate. Hence, the irradiation can be guided in accordance with the change in the location of the tumor.

The respiratory rate can also be determined by means of a camera which, for example, records the movement of the thorax; the respiratory rate can be determined on the basis of the up-and-down movement of the thorax.

Hence, it is possible to use information on the size, location and position of the tumor for more precise planning of the irradiation volume.

The FIGURE shows a flow diagram with steps of a method for planning irradiation and carrying out irradiation. In a step 101, MRI images are compiled and combined to form a 3D image. In a step 102, a plurality of 3D images over time is combined to form a 4D image (change of the recorded 3D object over time). In a step 103, a periodic movement over time is determined for an object in the 3D image. In particular, during the course of the period, a change to the size, the location and/or the position of the object is determined. Depending upon the periodic movement of the object, in a step 104, a radiation source is controlled in dependence on the movement of the object; for example, the radiation source is guided according to the periodic movement of the object—taking into account the size, location and/or position of the object.

Optionally, the step 104 can be performed by a unit that is separate from the unit for the determination of the periodic movement. In this case, the unit for the determination of the periodic movement provides parameters of the periodic movement as a function of a respiratory rate 105. The respiratory rate 105 can be determined by means of a breathing belt or by means of a camera (see above) and the radiation source can be guided according to the periodic movement taking into account the respiratory rate 105.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for determining a position of an object in a magnetic resonance scanner, comprising:
in a processor, compiling a plurality of temporally successive magnetic resonance images of the object; and
in said processor, automatically determining a periodic movement of the object from the compiled magnetic resonance images, and emitting an electrical signal representing said periodic movement.

2. A method as claimed in claim 1 wherein said periodic movement is determined based on respiration.

3. A method as claimed in claim 1 comprising determining said periodic movement based on a heartbeat.

4. A method as claimed in claim 1 comprising determining said periodic movement by modeling respiration.

5. A method as claimed in claim 1 comprising determining said periodic movement by executing a pattern recognition algorithm of said compilation of magnetic resonance images.

6. A method as claimed in claim 1 wherein said object is a tumor.

7. A method as claimed in claim 1 wherein said object is selected from the group consisting of a spherical region, an ellipsoidal region, a region containing a spherical region, a region containing an ellipsoidal region, a region within a spherical region, and a region within an ellipsoidal region.

8. A method as claimed in claim 1 comprising controlling a radiation source with said electrical signal, dependent on said periodic movement.

9. A method as claimed in claim 1 comprising compiling said temporally successive magnetic resonance images to compile three-dimensional images of the object and with a plurality of said three-dimensional images being compiled over time form a four-dimensional image of the object.

10. A method as claimed in claim 1 comprising determining said periodic movement dependent on a change selected from the group consisting of a change of a size of the object, a change of a location and alignment of the object, and a change of a position of the object.

11. An apparatus for determining a position of an object comprising:
a magnetic resonance scanner;
a control computer configured to operate said magnetic resonance scanner to acquire a plurality of temporarily successive images of an object situated in the magnetic resonance scanner, and to compile said plurality of temporally successive magnetic resonance images of the object; and
said processor being configured to automatically determine a periodic movement of the object from the compiled magnetic resonance images, and to emit an electrical signal representing said periodic movement.

12. An apparatus as claimed in claim 1 comprising a radiation source to which said electrical signal is applied, said radiation source being configured to control irradiation of the object dependent on said periodic movement represented in said electrical signal.

13. A non-transitory, computer-readable data storage medium encoded with programming instructions, said storage medium being loaded into a computer and said programming instructions causing said computer to:
compile a plurality of temporally successive magnetic resonance images of the object; and
automatically determine a periodic movement of the object from the compiled magnetic resonance images, and emit an electrical signal representing said periodic movement.
